(19) United States
(12) Patent Application Publication
(10) Pub. No.: US 2009/0117166 A1
(43) Pub. Date: May 7, 2009

(54) Sequential coupling of biomolecule layers to polymers

(76) Inventors: David Myung, Santa Clara, CA (US); Stayce Beck, Menlo Park, CA (US); Jaan Noolandi, Mountain View, CA (US); Christopher N. Ta, Saratoga, CA (US); Jennifer R. Cochran, Stanford, CA (US); Curtis W. Frank, Cupertino, CA (US)

Correspondence Address: LUMEN PATENT FIRM, 2345 YALE STREET, SECOND FLOOR, PALO ALTO, CA, 94306, US

(21) Appl. No.: 12/228,884

(22) Filed: Aug. 15, 2008

Related U.S. Application Data
(60) Provisional application No. 60/965,004, filed on Aug. 15, 2007.

(51) Int. Cl. A61F 2/04; A61K 9/10; A61K 38/39; A61K 38/30; A61K 38/18

(52) U.S. Cl. 424/422; 424/484; 514/12

(57) ABSTRACT

A bio-mimetic or bio-implantable material based on a sequential process of coupling biomolecule layers to a polymer layer is provided. In general, the material could be based on two or more biomolecule layers starting with one of the layers covalently linked to the polymer layer via cross-linkers and the other layers sequentially and covalently linked using cross-linkers to the previously added layer. The polymer layer could be a hydrogel or an interpenetrating polymer network hydrogel. The first layer of biomolecules could be a collagen type, fibronectin, laminin, extracellular matrix protein, or any combinations thereof. The second layer of biomolecules typically is a growth factor, protein or stimulant. The cross-linkers are either water soluble or insoluble bifunctional cross-linkers or azide-active-ester crosslinkers. The material and process as taught in this invention are useful in the field of tissue engineering and wound healing.

### CROSS-REFERENCE TO RELATED APPLICATIONS

This application claims priority from U.S. Provisional Application Nos. 60/965,004, filed on Aug. 15, 2007, which is incorporated herein by reference.

### FIELD OF THE INVENTION

The present invention relates generally to tissue engineering. More particularly, the present invention relates to materials and methods of sequentially coupled layers of biomolecules useful as tissue scaffolds and wound healing.

### BACKGROUND OF THE INVENTION

Wound healing in vivo is a sophisticated process involving interactions between migrating cells, their underlying matrix, and available growth factors. For a synthetic material to support this process on its surface, it must mimic the natural extracellular matrix (basement membrane), which contains a combination of proteins, growth factor (or growth-factor-like domains), and proteoglycans. Wound healing is especially important for epithelial wound healing of the skin or the surface of the cornea.

An important function of the cornea is to maintain normal vision by refracting light onto the lens and retina. This property is dependent in part on the ability of the corneal epithelium to undergo continuous renewal. Epithelial renewal is essential since it enables epithelial tissue to act as a barrier protecting the corneal interior from becoming infected by noxious environmental agents. Furthermore, the optical properties of the corneal epithelial surface are sustained through this renewal process. The rate of renewal is dependent on a highly integrated balance between the processes of corneal epithelial proliferation, differentiation, and cell death.

Disease or injury to the cornea is the second largest leading cause of blindness worldwide. Although treated in developed countries with transplants from donors, cornea transplants are unavailable in many parts of the world due to shortages of donors, or to cultural or religious barriers. In addition, the growing popularity of laser surgery is also reducing availability of corneas by making them unacceptable for donation.

Accordingly, an artificial cornea, which could restore the vision of more than 10 million people worldwide who are blind due to a diseased cornea, is needed in the art. However, for a synthetic material to support the process of wound healing on its surface, it must mimic the natural conditions as best as possible. Researchers have developed various kinds of techniques related to corneal prosthesis (see for example U.S. Pat. No. 6,689,165, U.S. Pat. No. 5,905,828 or US Patent Application 2007/0141105). The present invention further advances the art in a direction by providing a sequential coupling of layered biomolecules to promote epithelialization.

### SUMMARY OF THE INVENTION

The present invention provides a bio-mimetic or bio-implantable material based on a sequential process of layering biomolecules to a polymer layer. In general, the material could be based on two or more biomolecule layers starting with one of the layers covalently linked to the polymer layer via cross-linkers and the other layers sequentially and covalently linked to the previously added layer via cross-linkers.

In a preferred embodiment, the invention teaches two sequentially coupled layers of biomolecules linked to the polymer surface. The first layer of biomolecules is covalently linked to the polymer layer via a first set of cross-linkers, whereas a second layer of biomolecules is covalently linked to the first layer of biomolecules via a second set of cross-linkers. The polymer layer could be a hydrogel or an interpenetrating polymer network hydrogel. The first layer of biomolecules could include collagen type I, collagen type IV, collagen type V, collagen type VII, fibronectin, laminin, extracellular matrix protein, or any combinations thereof. The second layer of biomolecules could include epidermal growth factor, fibroblast growth factor, vascular endothelial growth factor, granulocyte colony stimulating growth factor, nerve growth factor, bone morphogenetic protein, transforming growth factor beta, activin, platelet derived growth factor, insulin like growth factor, hepatocyte growth factor, extracellular matrix protein or any combinations thereof. Typically, the first and second biomolecule layers contain different types of biomolecules. However, it is also possible to have two or more layers in the material that are of the same type of biomolecule, especially when the material is based on three or more sequentially coupled layers. The first or second sets of cross-linkers could be water soluble or insoluble bifunctional cross-linkers or azide-active-ester crosslinkers. Examples of azide-active-ester heterobifunctional crosslinkers include, but are not limited to N-5-Azido-2-nitrobenzoyloxysuccinimide, 6-(4-Azido-2-nitrophenylamino)hexanoic acid N-hydroxysuccinimide ester, N-Hydroxysulfosuccinimidyl-4-azidobenzoate, N-Succinimidyl-6-(4′-azido-2′-nitrophenylamino) hexanoate, or N-Sulfosuccinimidyl-6-(4′-azido-2′-nitrophenylamino) hexanoate.

In exemplary embodiments, the solution concentration of the first layer of biomolecules could be in the range of 0.01 mg/ml to 3 mg/ml and the solution concentration of the second layer of biomolecules could be in the range of 1 pg/ml to 1 mg/ml. The molecular weight of the first layer of biomolecules could be in the range of 50,000 to 500,000 and the molecular weight of the second layer of biomolecules could be in the range of about 3000 to 40,000. More generally speaking, the molecular weight of the first layer should be larger than the molecular weight of the second layer.

The material and process as taught in this invention are useful in the field of tissue engineering and wound healing in particular. For example, tissue scaffolds based on the invention can be applied in a large number of applications ranging from the eye, the mouth, the skin, the stomach, the gastrointestinal tract, the nose, the ear, the brain, the liver, the spine/vertebrae, intervertebral discs, the musculoskeletal system, and the cardiovascular system.

### BRIEF DESCRIPTION OF THE FIGURES

The present invention together with its objectives and advantages will be understood by reading the following description in conjunction with the drawings, in which:

FIG. 1 shows according to an embodiment of the invention a process and material based on sequential coupling of biomolecule layers. A first set of crosslinkers (e.g. heterobifunctional crosslinkers) link a first layer of biomolecules to a polymer surface. A second set of crosslinkers (e.g. heterobifunctional crosslinkers) links a second layer of biomolecules to the first layer of biomolecules. Additional layers can be added in a similar fashion. The crosslinking chemistry can be initiated/catalyzed by e.g. UV radiation (as shown), temperature, pH, enzymes, or the like.

FIG. 2 shows according to an embodiment of the invention the sequential coupling process of biomolecule layers. First, collagen is tethered to the hydrogels using photo-reactive azide chemistry. The azide linker is first linked to the hydrogel. The surface is then allowed to dry and then the hydrogel is exposed to UV. Next, the gel is soaked in a collagen/PBS solution allowing the collagen to tether to the hydrogel. After a series of washes to remove untethered collagen, EGF/azide mixture is added to the surface of the collagen-tethered hydrogel and the surface is allowed to dry. The hydrogel is then exposed to UV in e.g. 10 sec. pulses for 45 sec. allowing the EGF to tether to the collagen. A series of washes are then performed to remove untethered EGF and allow the hydrogel to swell.

FIG. 3 shows according to an embodiment of the invention experimental results of a corneal epithelial cells growing on collagen-coated tissue-culture polystyrene surfaces (TCPS) in the presence (row 2) or absence (row 1) of epidermal growth factor (EGF) in either the surrounding media (row 3) or non-specifically adsorbed (not covalently coupled) to the collagen surface (row 4).

FIG. 4 shows according to an embodiment of the invention experimental results of a corneal epithelial cells growing on collagen-coated tissue-culture polystyrene surfaces (TCPS) with epidermal growth factor (EGF) tethered using various UV exposure times.

FIG. 5 shows according to an embodiment of the invention immunofluorescent staining of cytokeratin 3/12 within corneal epithelial cells on collagen-coated TCPS in either (A) the absence of epidermal growth factor (EGF), (B) the presence of EGF in solution, and (C) the presence of surface-tethered EGF.

FIG. 6 shows according to an embodiment of the invention primary rabbit corneal epithelial cells grown on the hydrogel. When only exposed to collagen tethered to the hydrogel, we do not observe attachment and spreading (A). But when wild type EGF (B) is tethered we saw adherence and spreading of the epithelial cells. We also have a positive control with growth factors added into the media and the cells do indeed attach and spread in this case (C).

FIG. 7 shows according to an embodiment of the invention primary rabbit corneal fibroblast cells grown on the hydrogel. When there is only collagen on the hydrogel (A), the cells don't attach and spread, but when wild type EGF is present we see adherence and spreading of corneal fibroblast cells (B). We also have a positive control with growth factors added into the media and the cells do indeed attach and spread in this case (C).

FIG. 8 shows according to an embodiment of the invention a schematic of how a layered biomolecule surface can be used to improve the performance of tissue scaffolds, which require surface cell growth as well as 3-D tissue integration.

FIG. 9 shows according to an embodiment of the invention a schematic of how a layered biomolecule surface can be used implanted tissue scaffolds to regenerate damaged or diseased tissues.

### DETAILED DESCRIPTION

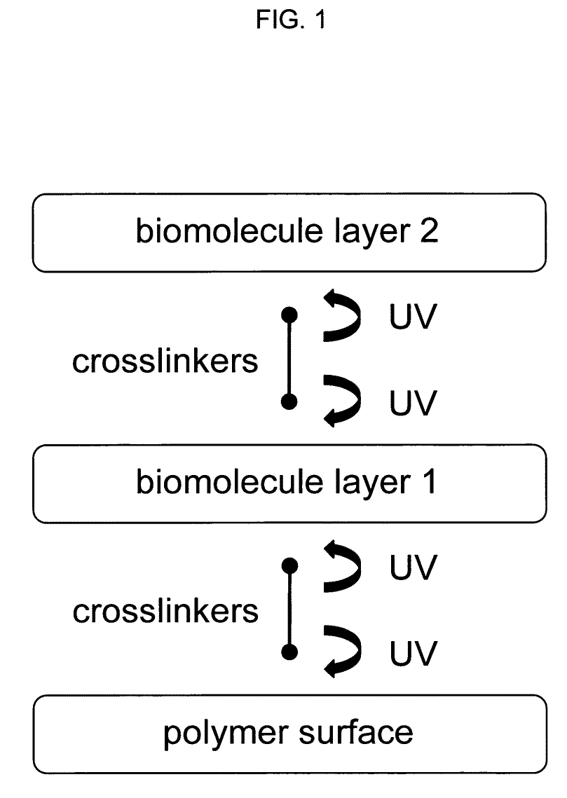

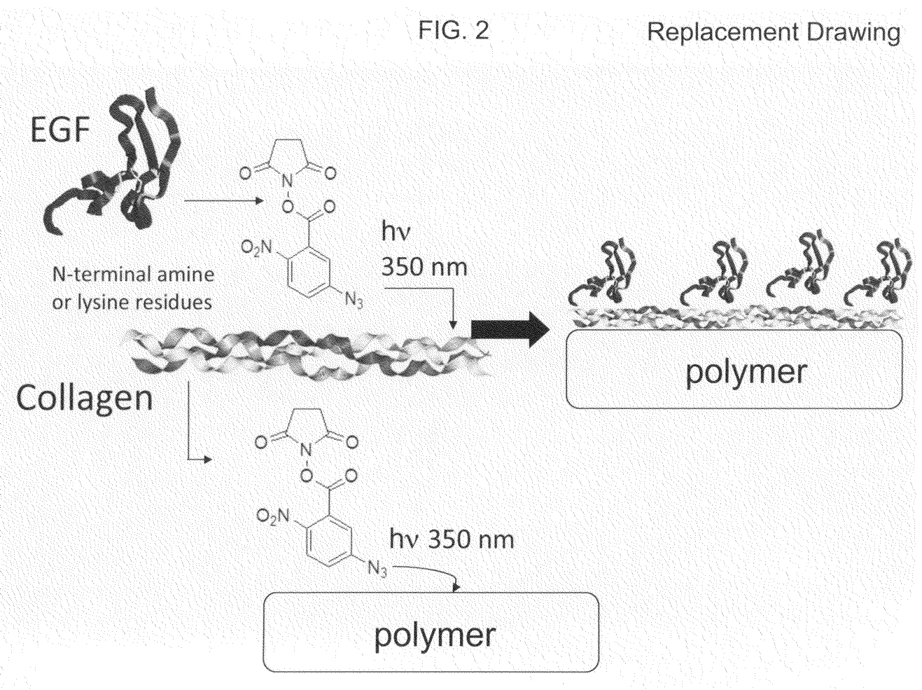

The present invention is a method (FIG. 1 and FIG. 2) for creating bioactive polymer surfaces through sequential coupling of biomolecule layers. Wound healing in vivo is a sophisticated process involving interactions between migrating cells, their underlying matrix, and available growth factors. For a synthetic material to support this process on its surface, it must mimic the natural extracellular matrix (basement membrane), which contains a combination of proteins, growth factor (or growth-factor-like domains), and proteoglycans. In vitro and in vivo experiments have shown that photochemical modification of non-adhesive PEG/PAA hydrogel surfaces with collagen type I can support the adhesion and multilayered growth of corneal epithelial cells. Presented in this invention is a method for sequentially coupling layers of cell adhesion-promoting biomolecules (e.g. matrix proteins) and cell proliferation promoting biomolecules (e.g. growth factors) to provide a more biomimetic synthetic basement membrane and will synergistically promote improved wound healing.

In one example, the invention is a process for creating a 2-layer matrix by deposition of biomolecules onto polymer surface. A first layer of biomolecules is deposited on to a polymer surface and allowed to adsorb or chemically bind to the polymer surface. A second layer of biomolecules with a reactive end group (or groups) is then deposited on top of the first layer of biomolecules. After exposure to UV light or another means of initiation, the second layer of biomolecules is then coupled to the first layer of biomolecules (protein) layer.

Alternatively, a two-step photochemical process can be used, in which the first layer of biomolecules (e.g. collagen) is first tethered to a hydrogel or polymer via azide-active ester photochemistry, followed by tethering of the second layer of biomolecules (epidermal growth factor, EGF) to the collagen, also via azide-active-ester photochemistry. Examples of azide-active-ester heterobifunctional crosslinkers used for the coupling strategy include, but are not limited to N-5-Azido-2-nitrobenzoyloxysuccinimide, 6-(4-Azido-2-nitrophenylamino)hexanoic acid N-hydroxysuccinimide ester, N-Hydroxysulfosuccinimidyl-4-azidobenzoate, N-Succinimidyl-6-(4′-azido-2′-nitrophenylamino) hexanoate, or N-Sulfosuccinimidyl-6-(4′-azido-2′-nitrophenylamino) hexanoate, While these examples provide typical embodiments, other chemical linking strategies can be used to link proteins to polymers or each other. Moreover, any combination of small molecules or biomolecules can be used for the different layers of biomolecules, including, but not limited to, drugs, chemicals, proteins, polypeptides, carbohydrates, proteoglycans, glycoproteins, lipids, and nucleic acids. Furthermore, the process of the invention is not limited to 2-layers, but can also be adapted to create 3 or more layers of the aforementioned biomolecules, containing either one type of biomolecule per layer or multiple types of biomolecules per layer.

In one example related to cell growth, a 2-layer bioactive surface was created on tissue culture polystyrene (TCPS) comprised of EGF bound to collagen on TCPS. First, a 0.3% solution of collagen type I (Inamed) diluted 1:25 in phosphate buffered saline (PBS) was incubated over the surface of 6-well TCPS plates for 1 hour. After removal of the collagen solution and washing with PBS, a layer of epidermal growth factor molecules was covalently tethered to the collagen-coated TCPS through azide-active-ester photochemistry.

First, 100 ug/mL of EGF (Invitrogen) was prepared in PBS (pH 7.4). One milligram of 5-azido-2-nitrobenzoic acid N-hydroxysuccinimide (NHS) ester was then dissolved in 1 mL of N,N-dimethylformamide. Next, 5.1 uL of this azide linker solution was added to 1 mL of the EGF solution to create an approximately 1:1 molar ratio between the EGF and the azide linker molecule. The reaction between the EGF free amines and the N—hydroxysuccinimide moiety in the linker was allowed to proceed overnight at room temperature on a shaker.

Substituted phenyl azides react with light (250-320 nm, 5 min) to generate aromatic nitrenes, which insert into a variety of covalent bonds. Upon UV irradiation, the phenyl azide group reacts to form covalent bonds with any surface containing carbon-hydrogen bonds. The solution of azide-functionalized EGF was evenly spread over the collagen-coated TCPS at various concentrations and then the PBS was evaporated under reduced pressure. The deposited surfaces were then exposed to UV light for various times (10-60 seconds) in 10-second pulses. Irradiated surfaces were thoroughly rinsed with PBS to remove any unreacted crosslinker/EGF from the surface.

Primary corneal epithelial cells isolated from rabbit corneas by an explant method known in the art and grown in keratinocyte serum-free media (Gibco-BRL) in the absence of epidermal growth factor were then cultured on these surfaces at a density of 4×10⁴ cells per well in 2 mL of culture medium. As positive and negative controls, cells were grown in the presence or absence of EGF in the media over collagen-only surfaces. Cells were also grown in the presence of EGF non-specifically adsorbed (but not covalently linked) to underlying collagen after 2 hours of incubation, as well as in the presence of media-based EGF that had been UV irradiated for 40 seconds. The cells were growth in culture for 1 week, and photographed in three high power fields every 24 hours for 3 days and then at 7 days.

Immunofluorescent staining of the marker for epithelial differentiation (cytokeratin 3/12) was accomplished by fluorescent microscopy. Epithelial cells grown on the various substrates were washed three times in Dulbecco's phosphate buffered saline and fixed for 5 min in 4% paraformaldehyde. The cells were permeabilized for 10 min with Triton X-100, and washed three additional times in phosphate buffered saline. Fixed and permeabilized cell samples were incubated in a 5% w/v bovine serum albumin solution for 10 min to block non-specific antibody binding. The samples were then incubated in a 1:1000 dilution of primary antibody (AE5 antibody against cytokeratin 3/12) within a moist chamber at room temperature for 90 min. This was followed by three washes in phosphate buffered saline and then incubation in 1:4000 solution of Alexa 488-labeled secondary antibody for 60 min in a dark, moist chamber at room temperature. A final three washes in phosphate buffered saline were followed by application of Vectashield with DAPI nuclear stain (Vector cat#: H-1200) and mounting of a coverslip. Samples were examined with a fluorescence-filtered Nikon phase contrast inverted microscope, or stored at 4° C. with light protection.

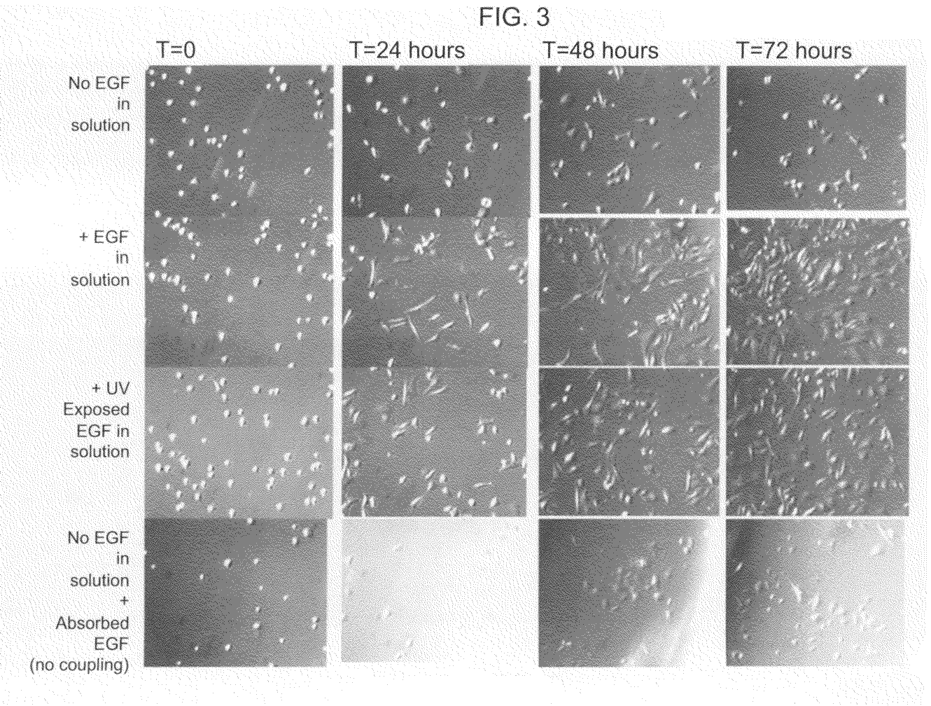

The results of these experiments are shown in FIGS. 3-5. Without EGF in the culture medium (FIG. 3, row 1), cell growth on collagen-coated TCPS remains sparse over 3 days. In contrast, in the presence of wild-type EGF (FIG. 3, row 2), the cells grow substantially better and more rapidly over 3 days. Short UV-exposure is not deleterious to the function of EGF, as cells appeared to have similar growth on collagen-coated TCPS in the presence of UV-exposed EGF and in the presence of nascent EGF (FIG. 3, row 3). Simple adsorption of EGF to the collagen is insufficient to promote synergistic cell growth, as cells shown in FIG. 3, row 4 show only minimal growth compared to the previous two cases.

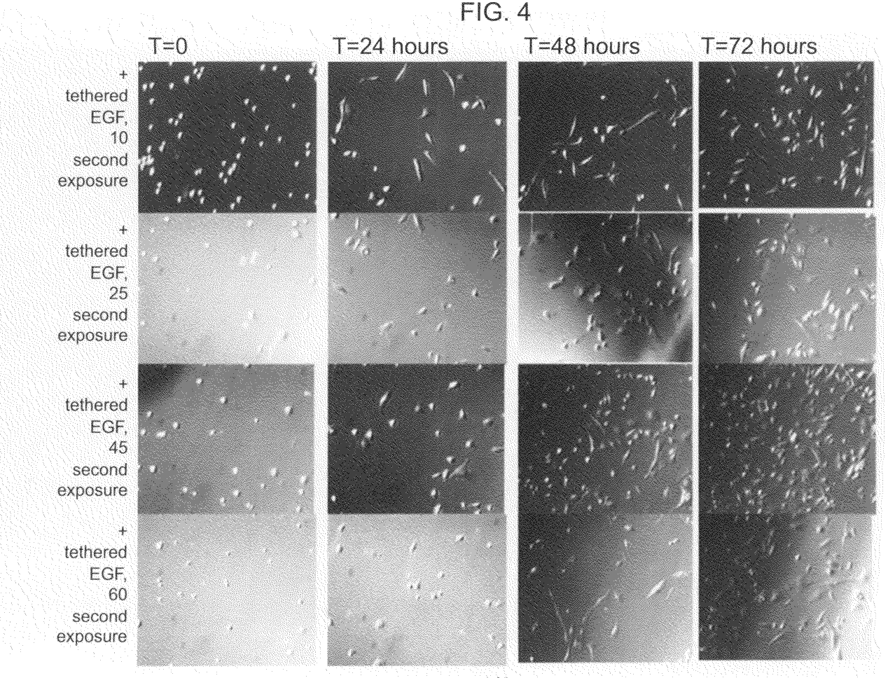

FIG. 4 suggests that successful covalent binding of EGF to the underlying collagen requires a balance between sufficient UV exposure to initiate tethering, and minimization of UV exposure to prevent protein denaturation. The EGF/collagen combination exerts its effect most prominently over 3 days when the deposited azide-functionalized EGF is exposed to UV for 45 seconds (FIG. 4, row 3) rather than 10, 25, or 60 seconds.

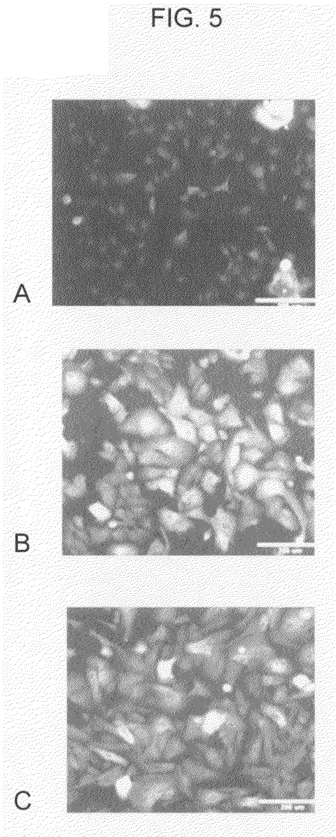

FIG. 5 shows results from immunofluorescent staining of cytokeratin 3/12 cells grown on EGF-tethered collagen-coated TCPS versus positive and negative controls, each for 7 days. Cells in both the positive control and tethered-EGF case had grown to confluence by day 7, while the negative control case yielded a sub-confluence cell layer. The absence of EGF results in minimal staining for cytokeratin 3/12, indicating that the cells are not able to remain differentiated in the absence of EGF. In contrast, EGF in solution (standard keratinocyte serum-free culture media) leads to strong epithelial differentiation of the cells, indicated by the wide-spread, diffuse, cytoplasmic green staining. Similarly, tethered EGF (without EGF in the surrounding culture media) also stains strongly, indicating robust epithelial differentiation of the cultured cells.

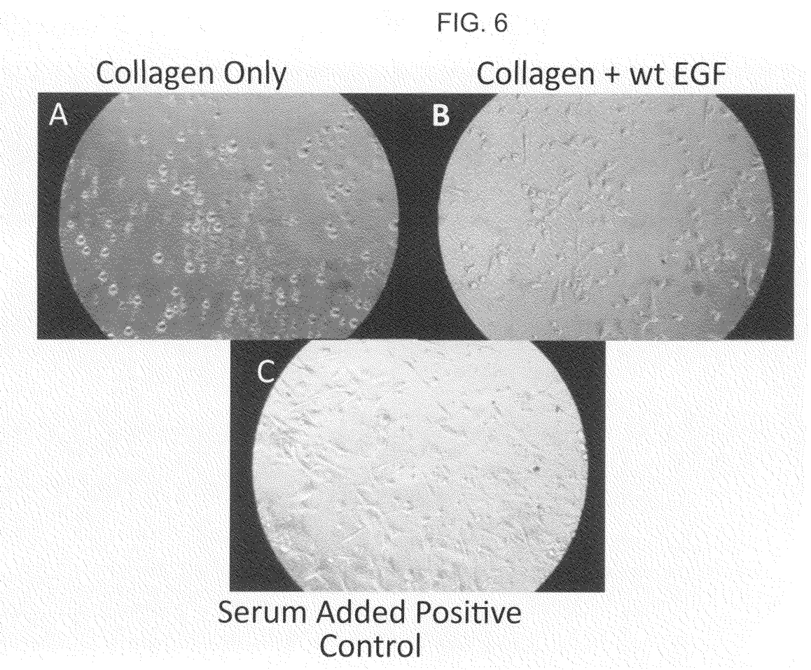

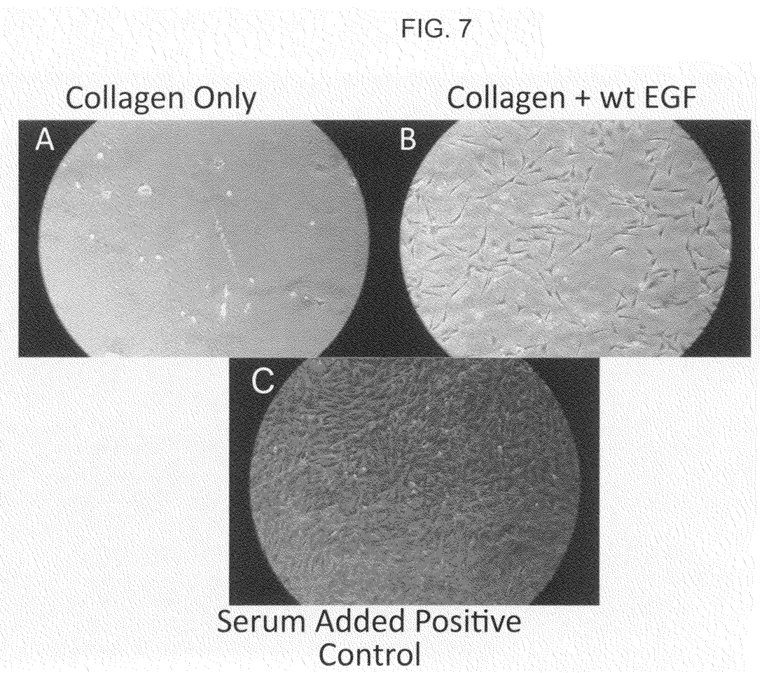

FIGS. 6 and 7 show that both primary cornea fibroblast and epithelial cells are only able to adhere and spread on the surface of the hydrogel when the hydrogel is tethered using a two step layering process with collagen and EGF. These results exhibit that the sequential tethering process with an extracellular matrix protein and growth factor can support surface epitheliallization on the PEG/PAA hydrogel.

The results show that a layered biomolecule surface combining an extracellular matrix protein and a growth factor stimulates synergistic cellular growth with normal cellular differentiation on a polymer surface. The processes described in this invention can be used to create layered surfaces of any combination of biomolecules to produce improved cell growth on polymer surfaces. Implantable tissue scaffolds can be created with this technology. For instance, a synthetic cornea based on a polymeric material or hydrogel can be surface modified using this layering method, creating a biomimetic surface on which epithelial and stromal cells can adhere and grow.

The material used in this invention can be either a polymer (including, but not limited to a polystyrene, polyester, acrylic, or cellulose) or a hydrogel, and includes both homopolymers (single networks), copolymers, and interpenetrating polymer networks (IPN) using any number of crosslinking methods (physical or chemical). Single network (homopolymer or copolymers) can include but are not limited to, polymers based on the following monomers: acrylonitrile, acrylic acid, acrylamide, hydroxyethyl acrylamide, N-isopropylacrylamide, methacrylic acid, 2-acrylamido-2-methylpropanesulfonic acid, 2-hydroxyethyl methacrylate, 2-hydroxyethyl acrylate, or derivatives and/or combinations thereof. Telechelic (end-functionalized) macromonomers of poly(ethylene glycol), such as poly(ethylene glycol)-diacrylate and poly(ethylene glycol)-dimethacrylate (or other end-linking functionalities) can also be used alone or in a copolymer with other monomers. In addition, poly(vinyl alcohol)-based hydrogels prepared by UV-crosslinking, freeze-thaw, or other means of crosslinking can be used. Biomacromolecules such as proteins (e.g. collagen), polysaccharides (e.g. chitosan), and other biomacromolecules such as hyaluronic acid, proteoglycans, glycoproteins, lipids, nucleic acids can be used alone, in combination, or in combination with synthetic monomers/polymers and crosslinking agents.

In one embodiment, the IPN contains a first polymer network, which is based on a hydrophilic telechelic macromonomer, and a second polymer network, which is based on a hydrophilic monomer. The hydrophilic monomer is polymerized and cross-linked to form the second polymer network in the presence of the first polymer network. Preferably, the first polymer contains at least about 50% by dry weight of telechelic macromonomer, more preferably at least about 75% by dry weight of telechelic macromonomer, and most preferably at least about 95% by dry weight of telechelic macromonomer. The telechelic macromonomer preferably has a molecular weight of between about 575 Da and about 20,000 Da. Mixtures of molecular weights may also be used.

In a preferred embodiment, the telechelic macromonomer is a vinyl-terminated poly(ethylene) glycol (PEG) such as PEG diacrylate or PEG dimethacrylate. Also preferably, the hydrophilic monomer in the second network is acrylic acid, acrylamide, hydroxyethyl acrylamide, N-isopropylacrylamide, methacrylic acid, 2-acrylamido-2-methylpropanesulfonic acid, 2-hydroxyethyl methacrylate, 2-hydroxyethyl acrylate, or derivatives and/or combinations thereof.

Variations include different polymers, different formulations of the polymers (weight ratio of the two or more polymer networks, crosslinking densities and methodologies, water content, and additional polymeric components), as well as variations in the size, shape, and implantation procedure of the polymer device. The choice of material can range from other hydrogel networks, to polymers like polyurethane and silicone as well as combinations of these with hydrophilic polymers. The interpenetrating polymer networks can be comprised of two or more networks or polymeric components (such as linear chains). Examples include but are not limited to a “triple” or even “quadruple” network or a double network interpenetrated with additional linear polymer chains. Fiber networks (such as electrospun nanofibers) as well as porous polymer or porous hydrogel structures may also be used.

Target organs include, but are not limited to, the eye (e.g. glaucoma, or diseases of the cornea or retina), the mouth, the skin, the stomach, the gastrointestinal tract, the nose, the ear, the brain, the liver, the spine/vertebrae, intervertebral discs, the musculoskeletal system, and the cardiovascular system. Small molecules or biomolecules attached by this layering technique include but are not limited to drugs, chemicals, proteins, peptides, polypeptides, glycoproteins, proteoglycans, growth factors (e.g. epidermal growth factor, fibroblast growth factor, transforming growth factor), immunoglobulins, nucleic acids, carbohydrates, lipids, lipoproteins, amino acids, and combinations thereof.

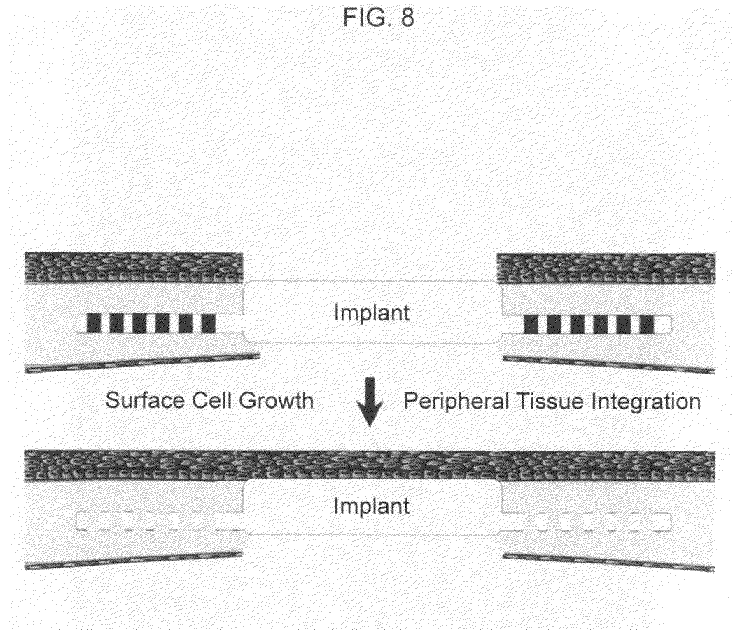

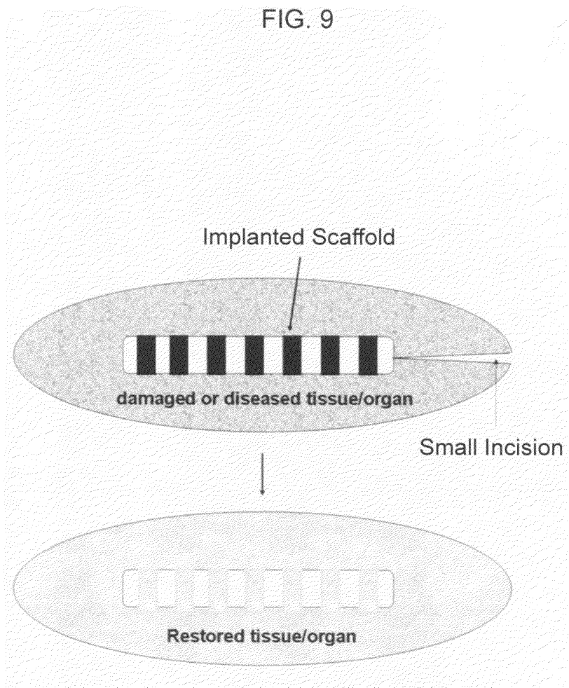

FIGS. 8 and 9 illustrate how the present invention can be used as a tissue scaffold in the body. Cell growth can be stimulated on layered biomolecule surfaces of polymers either two-dimensionally (on the outer surface) or three-dimensionally (along the inner and outer surfaces).

As a person of ordinary skill in the art will appreciate, various changes, substitutions, and alterations could be made or otherwise implemented without departing from the principles of the present invention. For example, referring back to the general concept of the invention as shown in FIGS. 1 and 2, the method may rely, for example, on (a) photoinitiated attachment of azidobenzamido peptides, (b) photoinitiated functionalization of hydrogels with an N-hydroxysuccinimide ester, maleimide, pyridyl disulfide, imidoester, active halogen, carbodiimide, hydrazide, or other chemical functional group, followed by reaction with peptides/proteins, or (c) chemoselective reaction of aminooxy peptides with carbonyl-containing polymers. Homofunctional crosslinkers could be used. For instance, if a large excess of homofunctional x-linker is used relative to the biomolecule, then the result is largely monomeric attachment at one end, leaving the other end free for attachment to another surface or moiety. In addition, polymeric tethers (such as poly(ethylene glycol) chains) can be used as intervening spacer arms between polymer surfaces and biomolecules and also between biomolecules. Finally, the aforementioned methods can be used in combination with each other to form the multilayered biomolecule surfaces. Accordingly, the scope of the invention should be determined by the following claims and their legal equivalents.

What is claimed is:

1. A method of making a bio-mimetic or bio-implantable material, comprising:
(a) providing a polymer layer;
(b) covalently linking a first layer of biomolecules to said polymer layer via a first set of cross-linkers; and
(c) covalently linking a second layer of biomolecules to said first layer of biomolecules via a second set of cross-linkers.

2. The method as set forth in claim 1, wherein said polymer layer is a hydrogel or an interpenetrating polymer network hydrogel.

3. The method as set forth in claim 1, wherein said first layer of biomolecules comprises collagen type I, collagen type IV, collagen type V, collagen type VII, fibronectin, laminin, extracellular matrix protein, or any combinations thereof.

4. The method as set forth in claim 1, wherein said second layer of biomolecules comprises epidermal growth factor, fibroblast growth factor, vascular endothelial growth factor, granulocyte colony stimulating growth factor, nerve growth factor, bone morphogenetic protein, transforming growth factor beta, activin, platelet derived growth factor, insulin like growth factor, hepatocyte growth factor, extracellular matrix protein or any combinations thereof.

5. The method as set forth in claim 1, wherein said first or said second sets of cross-linkers are water soluble or insoluble bifunctional cross-linkers or azide-active-ester crosslinkers.

6. The method as set forth in claim 1, wherein the solution concentration of said first layer of biomolecules is in the range of 0.01 mg/ml to 3 mg/ml and the solution concentration of said second layer of biomolecules is in the range of 1 pg/ml to 1 mg/ml.

7. The method as set forth in claim 1, wherein the molecular weight of said first layer of biomolecules is in the range of 50,000 to 500,000 and the molecular weight of said second layer of biomolecules is in the range of about 3000 to 40,000, or wherein the molecular weight of said first layer is larger than the molecular weight of said second layer.

8. The method as set forth in claim 1, wherein said first and said second biomolecules are different types of biomolecules.

9. The method as set forth in claim 1, further comprising covalently linking one or more additional layers of biomolecules in between said first layer of biomolecules and said second layer of biomolecules, wherein each one of said additional layers of biomolecules are covalently linked with each other and with said first and second layers of biomolecules via their own respective set of cross-linkers.

10. The method as set forth in claim 9, wherein said layers of biomolecules are different types of biomolecules.

11. A material, comprising:
(a) a polymer layer;
(b) a first layer of biomolecules covalently linked to said polymer layer via a first set of cross-linkers; and
(c) a second layer of biomolecules covalently linked to said first layer of biomolecules via a second set of cross-linkers.

12. The material as set forth in claim 11, wherein said polymer layer is a hydrogel or an interpenetrating polymer network hydrogel.

13. The material as set forth in claim 11, wherein said first layer of biomolecules comprises collagen type I, collagen type IV, collagen type V, collagen type VII, fibronectin, laminin, extracellular matrix protein, or any combinations thereof.

14. The material as set forth in claim 11, wherein said second layer of biomolecules comprises epidermal growth factor, fibroblast growth factor, vascular endothelial growth factor, granulocyte colony stimulating growth factor, nerve growth factor, bone morphogenetic protein, transforming growth factor beta, activin, platelet derived growth factor, insulin like growth factor, hepatocyte growth factor, extracellular matrix protein or any combinations thereof.

15. The material as set forth in claim 11, wherein said first or said second sets of cross-linkers are water soluble or insoluble bifunctional cross-linkers or azide-active-ester crosslinkers.

16. The material as set forth in claim 11, wherein the solution concentration of said first layer of biomolecules is in the range of 0.01 mg/ml to 3 mg/ml and the solution concentration of said second layer of biomolecules is in the range of 1 pg/ml to 1 mg/ml.

17. The material as set forth in claim 11, wherein the molecular weight of said first layer of biomolecules is in the range of 50,000 to 500,000 and the molecular weight of said second layer of biomolecules is in the range of about 3000 to 40,000, or wherein the molecular weight of said first layer is larger than the molecular weight of said second layer.

18. The material as set forth in claim 11, wherein said first and said second biomolecules are different type of biomolecules.

19. The material as set forth in claim 11, further comprising covalently linking one or more additional layers of biomolecules in between said first layer of biomolecules and said second layer of biomolecules, wherein each one of said additional layers of biomolecules are covalently linked with each other and with said first and second layers of biomolecules via their own respective set of cross-linkers.

20. The material as set forth in claim 19, wherein said layers of biomolecules are different types of biomolecules.
